(19) United States
(12) Patent Application Publication
(10) Pub. No.: US 2002/0111384 A1
(43) Pub. Date: Aug. 15, 2002

(54) Method of treating alzheimer's disease

(76) Inventors: Vickie L. Boudrie, Bradenton, FL (US); Craig E. Muirhead, Charlotte, MI (US)

Correspondence Address: Price, Heneveld, Cooper DeWitt & Litton, 695 Kenmoor, SE, Post Office Box 2567, Grand Rapids, MI, 49501, US

(21) Appl. No.: 09/977,533

(22) Filed: Oct. 15, 2001

Related U.S. Application Data
(60) Provisional application No. 60/240,744, filed on Oct. 16, 2000.

(51) Int. Cl. A61K 31/198

(52) U.S. Cl. 514/567; 514/646

(57) ABSTRACT

A method of treatment regimen for the treatment of Alzheimer's disease symptomology utilizing as an effective amount of a levodopa alone or in combination with an effective amount of carbidopa to reduce the progression of Alzheimer's disease while improving memory, cognitive functioning, and ability to perform daily living activities during the course of treatment.

### CROSS-REFERENCE TO RELATED APPLICATION

This application claims priority under 35 USC §119(e) on U.S. provisional application Serial No. 60/240,744, filed Oct. 16, 2000, the entire disclosure of which is incorporated herein by reference.

### FIELD OF THE INVENTION

The present invention relates to the treatment of Alzheimer's disease.

### DESCRIPTION OF THE RELATED ART/BACKGROUND INFORMATION

Alzheimer's disease is a common and complex disorder characterized by adult-onset progressive dementia. Generally, Alzheimer's disease is a degenerative disease of the brain from which there is no recovery. Slowly and inexorably, the disease attacks nerve cells in all parts of the cortex of the brain, as well as some of the surrounding structures and tissues, thereby impairing a person's ability to govern emotions, recognize errors and patterns, coordinate movement, and remember stored cognitive function. During the final stages of the disease, a patient suffering from Alzheimer's loses all memory and mental functioning to the point of complete central nervous system (CNS) collapse and cessation of regulated circulatory and respiratory function. T. D. Bird, MD, “Alzheimer Overview,” GeneClinics, 1999, p. 1.

Alzheimer's disease is the most common cause of dementia in North America and Europe. The prevalence of the disease increases with age. Approximately 10 percent of all persons over the age of 70 have significant memory loss and more than half of these individuals have Alzheimer's disease. The prevalence of dementia in individuals over the age of 85 is estimated to be about 25-45%. Because of increasing longevity, the occurrence of Alzheimer's disease in the elderly presents a tremendous medical, economic and social problem facing the health care industry today.

About half of the people in nursing homes and almost half of all people over age 85 have Alzheimer's disease. It is now the fourth leading cause of death in elderly adults. Almost 4 million Americans have Alzheimer's disease, and unless effective methods for prevention and treatment are developed by the pharmaceutical and medical industries, it will reach epidemic proportions by the middle of the next century, afflicting between 8 and 14 million people. T. D. Bird, MD, “Alzheimer Overview,” GeneClinics, 1999, p. 2.

The disease usually begins after age 65, and the risk of Alzheimer's disease goes up with age. While younger people may also suffer from Alzheimer's disease, it is much less common. About 3 percent of men and women ages 65-74 have Alzheimer's disease. Yet, Alzheimer's disease is not a normal part of the aging process. Alzheimer's disease is characterized by a gradual loss of memory, decline in the ability to perform a routine task, disorientation, difficulty in learning, loss of language skills, impaired judgement and ability to plan, and personality changes. Over time, these changes become so severe and impairing that they interfere with an individual's daily functioning, resulting eventually in death.

Typically, Alzheimer's disease begins with subtle and poorly recognized failure of memory. The early symptoms of Alzheimer's disease may be overlooked because such symptoms resemble signs of natural aging. These symptoms include forgetfulness, loss of concentration, unexplained weight loss and motor problems, including mild difficulties in walking. In healthy individuals, similar symptoms can result from fatigue, grief or depression, illness, vision or hearing loss, the use of alcohol with certain medications, or simply the burden of too many details to remember at once. Accompanying sensory problems, such as hearing loss and a decline in reading ability, as well as general physical debility indicate a short survival time. T. D. Bird, MD, “Alzheimer Overview,” GeneClinics, 1999, p. 1.

While the disease can last from 3-20 years after the onset of symptoms, the average duration is 8 years. Other symptoms include confusion, poor judgement, language disturbance, agitation, withdrawal, and hallucinations. Some patients may develop seizures, Parkinsonian-type features, decreased muscle tone, myoclonus, incontinence and mutism. Death usually results as the symptoms worsen over time. Michael Rebhan, Ph.D., “Alzheimer Fact Sheet,” 1998, p. 1; “Alzheimer's Disease,” Administration on Aging, 2000, p. 1.

About 75% of Alzheimer's disease occurs sporadically and about 25% of Alzheimer's disease can be divided into several subgroups on a genetic basis. Sporadic Alzheimer's disease includes most patients who meet the diagnostic criteria for Alzheimer's disease who have a negative family history. Onset can be any time in adulthood. The exact pathogenesis of the disease is unknown. A common hypothesis is that sporadic Alzheimer's disease is multifactorial and results from a combination of aging, genetic predisposition, exposure to one or more environmental agents such as head trauma, viruses, and/or toxins such as heavy metals including aluminum. T. D. Bird, MD, “Alzheimer Overview,” GeneClinics, 1999, p. 2.

Late onset familial Alzheimer's disease generally occurs in families which have multiple affected members, all of whom have onset of dementia, usually after the age of 65 or 70. This form of Alzheimer's disease represents at least 10-25% of all Alzheimer's patients. A third category, early onset familial Alzheimer's disease, refers to families in which multiple cases of Alzheimer's disease occur and the mean age of onset is before the age of 65 years. Generally, those with this form of Alzheimer's disease have an early onset and a positive family history for the disease. Ibid., pp. 2-3.

Finally, the last category of Alzheimer's disease is associated with Down's Syndrome. In prior art studies, patients with Down's Syndrome develop the neuropathological hallmarks of Alzheimer's disease after the age of 40. More than half of such individuals also show, if carefully observed or tested, clinical evidence of cognitive decline. It is presumed that this lifelong cognitive decline is due to the over-expression of the amyloid precursor protein (APP) gene on chromosome 21 and the resultant overproduction of A-beta amyloid. Ibid., p. 4.

Clinically, Alzheimer's disease is a neuropathological disease. Recognized clinical signs include progressive dementia and cerebral cortical atrophy, which can be established by neuroimaging studies. In addition, neuropathological findings usually include microscopic A-beta amyloid neuritic plaques, intraneuronal neurofibrillary tangles and amyloid angiopathy. Studies within the prior art have indicated that Alzheimer's disease is associated with cerebral cortical atrophy, histological findings of beta amyloid plaques and findings of intraneuronal neurofibrillary tangles within the cortical regions of the brain. Ibid., p. 1.

Neurofibrillary tangles are tangled fibers, which are the damaged remains of microtubules, within the cortical region of the brain, that support the structure allowing the flow of nutrients through nerve cells (neurons). Beta amyloid (A beta) is an insoluble protein which is a fragment of a larger protein (APP). Prior art studies have shown that APP itself appears to be important in nerve protection. Should the enzyme involved in cutting APP into fragments of beta amyloid fail to function, APP has been shown within the prior art to form sticky patches called neuritic plaques, which decrease neuronal function and signal transmission within the brain. Generally, such neuritic plaques are found on the outside of nerve cells surrounded by debris of dying neurons.

In addition, high levels of beta amyloid have been associated in the prior art with reduced levels of the neurotransmitter acetylcholine. Neurotransmitters are chemical messengers in the brain that transmit various signals, messages, and neurochemical information within the various regions of the CNS. Acetylcholine is part of the cholinergic system, which is essential for memory and learning, and is progressively destroyed in patients suffering from Alzheimer's disease. Thus, it is believed within the prior art that beta amyloid in the form of neuritic plaques causes a decrease in the neurotransmitter, acetylcholine, leading to progression of Alzheimer's disease.

Nerve cells transmit impulses much like wires transmit electricity. But unlike wires, which are continuous filaments, nerve cells do not physically touch one another within the human body. They have microscopic gaps between each of the nerves, called synapses. Nerve impulses must jump these synapses and proceed on their way to continue the communication pathway. They do so with the help of special chemicals, called neurotransmitters, such as acetylcholine. As a nerve impulse passes through a nerve cell, it activates the release of neurotransmitters into the cell synapses, allowing the impulse to jump the gap and proceed along its way. Once the impulse crosses the synapse, special enzymes, such as acetocholinesterase, eliminate the neurotransmitter, leaving the cell and synapse ready to react to the next incoming impulse. “Cholinesterase Inhibitors: Helping Nerves Communicate,” Alzheimers.com™, 2000, p. 1.

During previous prior art studies of Alzheimer's disease, it was determined that Alzheimer's patients suffer a loss of acetylcholine from their synapses within the brain. As a result, cholinesterase inhibition in the prior art has yielded the most promising results to date in slowing the progression of the disease. Cognex (tacrine) and Aricept (donezepil) are both cholinesterase inhibitors available within the prior art, which increase the amount of acetylcholine available to synapses within the brain.

Beta amyloid is also believed to disrupt channels that carry sodium, potassium and calcium within the brain, which are important for creating electrical impulses within cranial neurons. If these channels are damaged, an imbalance can interfere with nerve function and signal transmission leading to further degenerative nerve disease.

The mainstay of Alzheimer's disease treatment within the prior art is generally supportive and disease symptomology is managed on an individual basis based upon a patient's disease progression. In general, affected patients eventually require assisted living arrangements in nursing homes as their cognitive ability and capability to manage daily activities decreases. Most drugs currently available for the treatment of Alzheimer's disease and those under investigation within the prior art are aimed at slowing progression, but there is no cure for the disease. T. D. Bird, MD, “Alzheimer Overview,” GeneClinics, 1999, p. 6.

However, for some people in the early and middle stages of the disease, a variety of prior art treatment modalities may alleviate some cognitive symptoms. Also, some a medications may help control behavioral symptoms of Alzheimer's disease, such as sleeplessness, agitation, wandering, anxiety and depression. Treating these symptoms often makes patients more comfortable and their care easier for caregivers assisting this type of patient population. Michael Rebhan, Ph.D., “Alzheimer Fact Sheet,” 1998, p. 3.

To date, only Cognex® (tacrine), Aricept® (donezepil) and Exelon® (rivastigmine) have been approved for the treatment of Alzheimer's disease by the United States Food and Drug Administration (FDA). All of these drugs work by increasing the brain's supply of acetylcholine, a neurotransmitter communication chemical that is deficient in people with Alzheimer's disease. A fourth drug, galantamine (Reminyl®), is currently under FDA review for the treatment of Alzheimer's disease. “Is There a Treatment for Alzheimer's Disease,” Alzheimer's Association, 2000, p. 1.

Donezepil appears to be better tolerated than tacrine, and more effective in improving mental functioning for more people than tacrine. Typical side effects of both of these drugs, however, include nausea and diarrhea. In addition, donezepil and tacrine can cause hepatotoxicity in patients receiving these medicinal agents. Benefits from these cholinergic system based drugs can be seen with mild to moderate improvement in cognitive function in patients suffering from Alzheimer's disease. Yet, cognitive functioning in these patients decreases as these types of drugs are discontinued from a patient's therapeutic protocol due to side effects of the medication over time.

Other cholinergic protective drugs currently available include metrifonate (Promem®), and physostigmine (Synapton®). These drugs, however, do have a high incidence of severe stomach and intestinal side effects, such as diarrhea and cramping, and again, only slow the progression of Alzheimer's disease, rather than cure it. Since no cholinergic protective drug will cure Alzheimer's, medicinal agents are currently only approved by the FDA for the treatment of mild to moderate Alzheimer's disease, but not as advanced stages.

Alternatives to cholinergic system based treatment modalities include non-steroidal anti-inflammatory drugs. Nonsteroidal anti-inflammatory drugs (NSAIDs) such as aspirin, ibuprofen, and naprosyn, have properties that block specific factors in the inflammatory response believed to play a major role in nerve-cell degeneration. However, long term use of NSAIDs can cause bleeding and ulcers in the gastrointestinal tract. Prior art treatment modalities have created combinations of NSAIDs and gastro-protective agents, such as diclofenac and misoprostol to reduce this risk considerably. Still, such NSAIDs can, over long periods of treatment use, create a concern for bleeding and ulceration in the gastrointestinal tract. Newer NSAIDs called COX-2 inhibitors (Vioxx®, Celebrex®) are also currently under investigation as to their nerve-protecting properties with minimal side effects to the gastrointestinal tract.

As an alternative to nonsteroidal anti-inflammatory agents, a variety of other drug products are being investigated within the prior art as to their effects upon Alzheimer's disease progression. Prior art studies for Vitamin E and selegiline have shown that these agents can slow the progression of Alzheimer's disease in its early stages. Prior art sources have suggested that Vitamin E, as an antioxidant, slows nerve cell degeneration while selegiline, acting as a selected monoamine oxidase B (MAO-B) inhibitor, increases the amount of acetylcholine available to cholinergic receptors within the brain, thus decreasing Alzheimer's progression.

However, Vitamin E has been shown within the prior art to cause nausea, cramping, and may increase the risk of bleeding in patients having coagulation abnormalities or who are taking blood thinning drugs. In addition, selegiline may cause a number of side effects including orthostatic hypotension, drowsiness, dizziness, sexual dysfunction, and insomnia.

Natural based herbal products have also been suggested for their ability to treat Alzheimer's disease progression. Ginkgo biloba is a common herb that has antioxidant properties and appears to increase blood flow to the brain. For example, prior art studies have suggested that gingko biloba may slightly improve the memory of Alzheimer's patients, although it is not clear that the improvement is clinically significant. Another herbal product, HupA is being investigated as to its ability to protect nerve cells from the harmful effects of beta amyloid, which may slow the progression of Alzheimer's disease.

Prior art studies are currently investigating hormone based treatment modalities for Alzheimer's disease as well. Melatonin, a naturally occurring hormone secreted by the pinial gland, is currently being studied as to its effects upon sleep disruption, one of the most common behavioral problems occurring in a majority of Alzheimer's disease sufferers. Estrogen replacement therapy is also currently being investigated for its effects upon Alzheimer's disease progression, but to date no significant clinical outcomes have been reported.

Lastly, prior art investigations are being completed as to the effect of neuroprotective and growth hormone based agents for Alzheimer's treatment. Neurotrophin™ is being investigated as to its growth factor effect upon nerves within the brain and how such effects upon neurology and behavioral function affect Alzheimer's disease progression. Further, propentofylline is currently being investigated for its effects upon Alzheimer's progression by enhancing metabolism in the brain. Finally, astaxanthin, a nutricutical with strong antioxidant properties, is being studied within the prior art as to its effects upon nerve degeneration in relation to the treatment of Alzheimer's disease.

Yet, because there is no cure for Alzheimer's disease symptomology to date, there is a need within the prior art for a composition and method of treatment regimen which is cost effective, slows progression of the disease with minimal side effect potentials, while positively increasing memory, cognitive function, and ability to perform daily living activities.

### SUMMARY OF THE INVENTION

A pharmaceutical composition and method of treatment regimen for the treatment of Alzheimer's disease symptomology which slows the progression of the disease through the use of an effective amount of levodopa alone or preferably in combination with an effective amount of carbidopa. The present invention reduces the negative effects of Alzheimer's disease symptomology by slowing the progression of the disease while increasing memory, cognitive function, and ability to perform daily living activities in patients being treated for Alzheimer's with the present invention in a cost effective manner.

By reducing the negative effects of Alzheimer's disease symptomology progression, the present invention also decreases the risk of injury to patients being treated for Alzheimer's disease with the present invention and may also allow for increased use of outpatient treatment settings for early disease stage patient populations, which in turn decreases the overall health care cost in treating such patient populations.

These and other features, advantages and objects of the present invention will be further understood and appreciated by those skilled in the art by reference to the following specification, claims and appended drawings.

### DETAILED DESCRIPTION OF THE PREFERRED EMBODIMENT

The preferred embodiment comprises of a pharmaceutical composition and method of treatment regimen containing an effective amount of a levodopa component alone or in combination with an effective amount of a carbidopa component in the treatment of Alzheimer's disease symptomology to slow the disease progression while increasing memory, cognitive function and ability to perform daily living activities. The levodopa component utilized in the present invention consists of an effective amount of levodopa, derivatives thereof, and pharmaceutically acceptable salts thereof, alone or in combination with an effective amount of the carbidopa component, which consists of an effective amount of carbidopa, derivatives thereof, and pharmaceutically acceptable salts thereof.

The effective amount of the levodopa component of the preferred embodiment is from about 0.5 g to about 8 g per day, preferably 2 to 5 g daily, and most preferably 0.5 to 1 g daily, divided into individual doses. Generally, doses of the levodopa component are divided into two or more doses and are preferably given with food to increase the absorption and bioavailability of the levodopa component. Dosage of the levodopa component should be adjusted gradually in increments not exceeding 0.75 g per day and preferably every 3-7 days, as tolerated by the patient undergoing treatment with the present invention. Furthermore, dosages generally do not exceed 8 g per day, but it should be understood by those of ordinary skill in the art that the dose of the levodopa component of the present invention may go higher than 8 g per day for exceptional patients requiring dosages exceeding this general dosage limit.

The effective amount of the carbidopa component of the preferred embodiment is from about 10 mg to about 200 mg per day, more preferably 50 to 150 mg per day and most preferably 70 to 100 mg per day given in divided doses.

The levodopa component of the preferred embodiment can be used separately, but administered contemporaneously and in combination with the carbidopa component. Either of the components can be given via a singular pharmaceutically acceptable dosage form or a combination of each of the components as immediate release or controlled release dosage form. Contemporaneously means the two agents can be administered separately over time, but have a combined effect together for their individual administrations. Suitable pharmaceutical dosage forms of the preferred embodiment include, but are not limited to, tablets, capsules, caplets, dose-paks, solutions, syrups, suppositories, transdermal applications, creams, lotions, emulsions, powders and the like. Preferred dosage forms for the present invention include tablets, caplets, capsules, dose-paks, solutions, and transdermal applications, with a tablet, caplet, or capsule being the most preferred.

The levodopa and carbidopa components of the composition and method of treatment of the preferred embodiment can be administered to the human body via a variety of medically and pharmaceutically acceptable administration routes. Those routes include, but are not limited to, the oral, rectal, intravenous, intradermal, subcutaneous, cutaneous, intramuscular, buccal, transdermal, and other pharmaceutically acceptable and medically acceptable routes of administration for the human body. Preferred routes of administration for the preferred embodiment are the oral, rectal, intravenous and intramuscular routes, with the oral route being most preferred. By confining the pharmaceutical medicaments of the preferred embodiments in a composition and method of treatment regimen for Alzheimer's disease, the preferred embodiment achieves significant slowing and reduction of the negative memory, cognitive functioning, and ability to perform daily living activity outcomes observed in patients suffering from Alzheimer's disease. Further, the preferred embodiment reduces the risk of injury to patients suffering from the disease by decreasing the negative disease symptomology outcomes of Alzheimer's disease progression. In addition, the preferred embodiment may allow for the treatment of Alzheimer's disease patients in an outpatient setting more frequently, thus reducing the overall cost of healthcare.

Senemet® by DuPont is a commercially available drug used for treating Parkinson's disease, which comprises a combination of levodopa and carbodopa. The available combinations include:

| | | |
|---|---|---|
| Sinemet ® | 10/100 | 10 mg carbidopa, 100 mg levodopa |
| Sinemet ® | 25/100 | 25 mg carbidopa, 100 mg levodopa |
| Sinemet ® | 25/250 | 25 mg carbidopa, 250 mg levodopa |
| Sinemet ® | CR | A sustained release version available as 25 mg |
| | | carbidopa, 100 levodopa, or 50 mg carbidopa, |
| | | 200 mg levodopa. |

Thus, Sinemet® provides a convenient source for the levodopa and carbidopa combination, which is preferred, and found useful for treating Alzheimer's.

Those skilled in the art will appreciate and be able to adjust the dose, dosing interval and dosing length/treatment period of the levodopa and carbidopa components of the preferred embodiments in the treatment of Alzheimer's disease symptomology, based on the clinical response and therapeutic value required to reduce or prevent the negative disease symptomology outcomes for patients undergoing such treatment. One skilled in the art will be able to adjust and appreciate the dose, dosing interval and length of treatment with the levodopa and carbidopa components of the preferred embodiment based on the liver and kidney function of the patient and ability to reduce the dose of the levodopa component in relation to the carbidopa component.

For example, as the dose of the carbidopa component is increased within the preferred embodiment, the levodopa component of the preferred embodiment may be decreased in relation to the carbidopa component. Prior art medical and pharmaceutical studies have shown that carbidopa has the capability to increase the amount and enhance the effect of levodopa within the human brain to increase the amount of acetylcholine available to neuronal receptors.

It should also be understood by those of ordinary skill in the art that the preferred embodiment can also be used in combination with other medically and pharmaceutically acceptable agents used in the treatment of Alzheimer's disease. For example, the preferred embodiment may also be used in combination with other cholinergic system based treatment modalities such as cognex, hormonal based therapies, such as estrogen replacement therapy, herbal based therapies such as ginkgo biloba, and vitamin and mineral supplementation therapies such as increased Vitamin E therapy. Thus, the preferred embodiment can be used to treat Alzheimer's disease symptomology alone, but can also be added to other currently available prior art treatment modalities to enhance the effects of those prior art treatments. In doing so, the present invention has the ability to enhance currently available treatment modalities to achieve increased treatment benefits to patients suffering from Alzheimer's disease.

Example: Treatment of Alzheimer's with Preferred Embodiment

The following observational example illustrates the outcome associated with use of the levodopa and carbidopa components of the preferred embodiments in treating Alzheimer's disease.

A female patient was suffering from increased difficulty in performing daily living activities and diminished cognitive functioning to a point requiring admission to an advanced 24-hour skilled nursing facility occurred on Aug. 3, 1999. At that time, the patient was assessed as having significant cognitive loss, limited ability to ambulate properly, and extensive linguistic and attention deficits. Following initial assessment, the patient was started on speech therapy five times weekly for eight weeks to increase the attention span and develop effective communication with staff and family to utilize during activities of daily life. However, over the eight-week period, the patient suffered from such extensive attention deficit that speech therapy proved ineffective to adequately increase the attention span and develop effective communication for the patient, staff and family during activities of daily life. Due to such a deficit, review of the patient's charts and records produced a free admission screening which evidenced severe patient mental illness or dementia, including significant disturbances in thought, conduct, emotions, and judgement. Further, the pre-admission screening of Jul. 14, 1999, also showed the patient at that time was unable to attend to a structured task for more than one minute before redirection was required. In addition, at that time, the patient was characterized as having confused speech and poor organization skills requiring further diagnosis and treatment.

Following discovery of the pre-admission screening assessment and diagnostic evaluation, the patient must start it upon repetitive memory testing and evaluation. However, such repetitive memory testing to increase cognitive functioning proved ineffective such that the patient was observed from November 1999 until February 2000 as deteriorating in cognitive function, memory and ability to perform activities of daily life to a point requiring around the clock physical, mental and emotional care. In addition, patient was observed on Feb. 24, 2000 by the attending physician as having diminished ambulation capability leading to the diagnosis of Parkinson's disease. As a result, the attending physician added Parkinson's disease as a diagnosis to the patient's medical record and at that time started patient upon Sinemet 25-100, one-half tablet three times a day for two weeks, and increased thereafter to one tablet three times a day for treatment of the Parkinsonian syndrome. Also at that time, the attending physician noted that the patient's mental condition was properly diagnosed as Alzheimer's disease based.

Over the course of two months, the patient was assessed by staff and family with regard to the Parkinsonian syndrome. The patient was given Sinemet 25-100 one-half tablet three times a day with meals for three weeks, and then increased to one tablet three times a day with meals thereafter. During this course of therapy, the patient was noted as having increased capability to ambulate, but also on March 9, March 13, April 1, April 8, and April 17 as having increased mental alertness and cognitive functioning. On Apr. 24, 2000, the patient was assessed for functional cognitive abilities as follows:

| | | Base Line | Current |
|---|---|---|---|
| 1. | Visual scanning task with 70% accuracy | 22% | 36% |
| 2. | Sorting task with 70% accuracy | 20% | 58% |
| 3. | Answering yes/no questions with 70% | 25% | 79% |
| | accuracy | | |
| 4. | Attend to an activity for five minutes | 5 minutes | 4.5 minutes |
| | minimum | | |

As a result of this assessment, the family inquired to attending physician as to patient's increased mental alertness and cognitive functioning. Following physician assessment, the patient's Parkinsonian syndrome therapy was increased to 1½ tablets three times a day for three weeks with meals to improve the Parkinsonian syndrome, but also to assess the ability of the Sinemet agent to improve the patient's cognitive abilities.

On Apr. 27, 2000, the patient was noted by staff as having increased mental alertness, increased cognitive functioning, and ability to walk without assistance at a greater capability than previously noted. Further, staff noted that the patient was very awake and appeared to have increased memory to perform activities of daily life. At that time, the patient was placed upon further mental stimulating abilities to develop further cognitive functioning. On follow-up assessments of May 2 and May 9, patient again exhibited increased mental alertness, improved memory and increased cognitive functioning to allow for further capabilities within the activities of daily life realm.

As can be seen from this observational example, it has been discovered that the use of an effective amount of levodopa alone or in combination with an effective amount of carbidopa reduces and slows the progression of Alzheimer's disease symptomology, causing increased memory, cognitive functioning and ability to perform activities of daily living than treatment modalities without the inclusion of the present invention.

The above description is considered that of the preferred embodiments only. Modification of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the Doctrine of Equivalents.

The invention claimed is:

1. A method for treating Alzheimer's disease comprising administering an effective amount of a levodopa component, alone or in combination with a carbidopa component.

2. The method of claim 1, wherein said levodopa component is a member selected from the group consisting of levodopa, derivatives thereof, and pharmaceutically acceptable salts thereof.

3. The method of claim 2, wherein said carbidopa component is administered in combination with said levodopa component, and said carbidopa component is a member selected from the group consisting of carbidopa, derivatives thereof, and pharmaceutically acceptable salts thereof.

4. The method of claim 3, wherein said effective amount of said levodopa component is from about 0.5 g to about 8 g per day.

5. The method of claim 4, wherein said effective amount of said carbidopa component is about 10 mg to about 200 mg per day.

6. The method of claim 5, wherein said effective amount of said levodopa and said effective amount of said carbidopa are delivered to the human body via medically and pharmaceutically acceptable dosage forms.

7. The method of claim 6, wherein said medically and pharmaceutically acceptable dosage form is a member selected from the group consisting of tablets, capsules, caplets, dose-paks, solutions, syrups, suppositories, transdermal applications, creams, lotions, emulsions, powders, and other pharmaceutically acceptable dosage forms.

8. The method of claim 7, wherein said dosage form is a tablet.

9. The method of claim 6, wherein said effective amount of said levodopa and said effective amount of said carbidopa are delivered to the human body via a medically or pharmaceutically acceptable administration route.

10. The method of claim 9, wherein said medically and pharmaceutically acceptable administration route is a member selected from the group consisting of oral, rectal, intravenous, intradermal, subcutaneous, cutaneous, intramuscular, buccal, transdermal, and other pharmaceutically and medically acceptable administration routes to the human body.

11. The method of claim 10, wherein said medically and pharmaceutically acceptable administration route is the oral route.

12. The method of claim 1, wherein said carbidopa component is administered in combination with said levodopa component.

13. The method of claim 12, wherein said effective amount of said levodopa component is from about 0.5 g to about 8 g per day.

14. The method of claim 12, wherein said effective amount of said carbidopa component is about 10 mg to about 200 mg per day.
